(19) United States
(12) Patent Application Publication
(10) Pub. No.: US 2026/0275181 A1
(43) Pub. Date: Sep. 17, 2026

(54) HEAT TRANSFER MEDIUM

(71) Applicant: AGC Inc., Tokyo (JP)

(72) Inventors: Hidefumi SHIOTA, Tokyo (JP); Takafumi KAWAKAMI, Tokyo (JP); Satoshi KAWAGUCHI, Tokyo (JP); Yu TAKEUCHI, Tokyo (JP)

(73) Assignee: AGC Inc., Tokyo (JP)

(21) Appl. No.: 19/673,369

(22) Filed: May 11, 2026

Related U.S. Application Data
(63) Continuation of application No. PCT/JP2024/040395, filed on Nov. 14, 2024.

(30) Foreign Application Priority Data
Nov. 17, 2023 (JP) 2023-196150

(51) Int. Cl. C09K 5/10; H10P 72/00

(52) U.S. Cl. CPC C09K 5/10; H10P 72/0421

(57) ABSTRACT

The heat transfer medium of the present invention comprises a compound (A) represented by the following formula (1):

$${R}^{A} - O - {R}^{1} - O - {R}^{B} \qquad ( 1 )$$

wherein R^{A} is a perfluoroalkenyl group having 2 to 4 carbon atoms; R^{B} is a fluoroalkyl group having 2 to 4 carbon atoms or a fluoroalkenyl group having 2 to 4 carbon atoms; and R¹ is an alkylene group having 1 to 4 carbon atoms.

### TECHNICAL FIELD

The present invention relates to a heat transfer medium using a hydrofluoroether olefin (HFEO).

### BACKGROUND ART

Heat transfer media are used in various types of heat transfer means, such as temperature control of wafers in semiconductor production, cooling and heating of semiconductor devices and electronic parts, cooling of servers, and temperature control of heat pumps, heat pipes and thermostatic chambers.

Perfluorocarbon (PFC), from the viewpoint of its nonflammability, its low ozone depletion potential (ODP), and the like, has conventionally been widely used as the heat transfer media. PFC, however, has such problems as high global warming potentials (GWP) and high environmental impacts due to the greenhouse effect.

Hence, use of HFE (hydrofluoroether) having a low ODP, a low GWP and a lower environmental impact as an alternative heat transfer medium to PFC has been studied (see, for example, PTL1).

### CITATION LIST

### Patent Literature

PTL1: Japanese Translation of PCT International Application Publication No. 2007-524737

### SUMMARY OF INVENTION

### Technical Problem

For example, in a dry etching step using plasma in semiconductor production, there are cases where a heat transfer medium is used under the severe conditions of a high electric field and a high-temperature environment. Hence, for the heat transfer medium, not only the heat conduction, but also excellent electric characteristics such as high insulation and the like are demanded.

Also a heat transfer medium lower in the environmental load than HFE conventionally used is desired.

The present invention has been achieved in consideration of such a situation and has an object to provide a nonflammable heat transfer medium low in the environmental impact and high in the insulation.

### Solution to Problem

The present invention is based on such a finding that an HFEO having a structure in which hydrogen fluoride has been eliminated from an HFE can be used as a heat transfer medium low in the environmental impact and high in the insulation.

The present invention provides the following means.

[1] A heat transfer medium, comprising a compound (A) represented by the following formula (1):

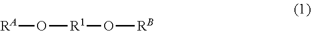

wherein R^{A} is a perfluoroalkenyl group having 2 to 4 carbon atoms; R^{B} is a fluoroalkyl group having 2 to 4 carbon atoms or a fluoroalkenyl group having 2 to 4 carbon atoms; and R¹ is an alkylene group having 1 to 4 carbon atoms.

[2] The heat transfer medium according to [1], wherein either one carbon atom constituting a carbon-carbon double bond in the perfluoroalkenyl group of R^{A} is bound to the adjacent oxygen atom.

[3] The heat transfer medium according to [1] or [2], wherein either one carbon atom constituting a carbon-carbon double bond in the fluoroalkenyl group of R^{B} is bound to the adjacent oxygen atom.

[4] The heat transfer medium according to [1] or [2], wherein R^{B} is a fluoroalkyl group having 2 to 4 carbon atoms and one hydrogen atom.

[5] The heat transfer medium according to [1], wherein the compound (A) is 1,2,3,3,3-pentafluoro-1-[2-(1,1,2,3,3,3-hexafluoropropoxy)ethoxy]-1-propene, 1,2,3,3,3-pentafluoro-1-[3-(1,1,2,3,3,3-hexafluoropropoxy)propoxy]-1-propene, or 1,2,3,3,4,4,4-heptafluoro-1-[2-(1,1,2,3,3,4,4,4-octafluorobutoxy)ethoxy]-1-butene.

[6] The heat transfer medium according to any one of [1] to [5], wherein the content of the compound (A) is 50% by mass or higher.

[7] The heat transfer medium according to any one of [1] to [6], wherein the heat transfer medium is used for cooling or heating a component member in a semiconductor production apparatus.

### Advantageous Effects of Invention

According to the present invention, there can be provided a nonflammable heat transfer medium low in the environmental load and high in the insulation.

### DESCRIPTION OF EMBODIMENTS

A heat transfer medium of an embodiment of the present invention (hereinafter, referred to also as the present embodiment) comprises a compound (A) represented by the following formula (1):

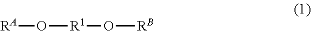

In the formula (1), R^{A} is a perfluoroalkenyl group having 2 to 4 carbon atoms; R^{B} is a fluoroalkyl group having 2 to 4 carbon atoms or a fluoroalkenyl group having 2 to 4 carbon atoms; and R¹ is an alkylene group having 1 to 4 carbon atoms.

The compound (A) is an HFEO. Since HFEO has a carbon-carbon double bond easily decomposable by active oxygen in a hydroxy radical or the like in the atmosphere, it is short in the atmospheric lifetime, low in GWP and low in the global environmental impact. The compound (A) is a fluorocompound, and has no flash point in the same way as HFE, and is nonflammable. The compound (A) is higher in the insulation than HFE widely used.

Accordingly, the heat transfer medium of the present embodiment is low in the environmental load and high in the insulation and nonflammable.

Then, the GWP is a value estimated by summing intensities over 100 years of the greenhouse effect (radiant energy given to the earth) per concentration when a substance of interest is emitted in the atmosphere, with respect to a reference intensity of carbon dioxide assigned a GWP of 1. The GWP of the compound represented by the formula (1) is determined as an estimated value based on its chemical structure from GWP values of known fluorine-based heat transfer media.

The ODP is a numerical value represented as a relative value of an amount of ozone depletion imposed to the ozone layer when a substance of interest is emitted in the atmosphere, with respect to a reference amount of ozone depletion per 1 kg of CFC-11 (trichlorofluoromethane) assigned an ODP of 1.0. The ODP of HFEO, since containing no halogen other than fluorine, is 0 as in HFE.

The dielectric breakdown voltage (in the case of the electrode gap of 2.5 mm) of the heat transfer medium is, from the viewpoint of stable use thereof in a high-electric field environment, preferably 20 kV or higher, more preferably 30 kV or higher and still more preferably 40 kV or higher.

The heat capacity (25° C.) per unit volume of the heat transfer medium is, from the viewpoint of efficient heat transfer, preferably 1.5 kJ/(L·K) or higher, more preferably 1.6 kJ/(L·K) or higher and still more preferably 1.7 kJ/(L·K) or higher.

Then, the heat conductivity (25° C.) of the heat transfer medium is, from the viewpoint of efficient heat transfer, preferably 50 mW/(m·K) or higher, more preferably 60 mW/(m·K) or higher and still more preferably 70 mW/(m·K) or higher.

The content of the compound (A) in the heat transfer medium of the present embodiment is, from the viewpoint of making the heat transfer medium to be a stable heat transfer medium capable of fully exhibiting the characteristics of the compound, preferably 50% by mass or higher, more preferably 80% by mass or higher, still more preferably 95% by mass or higher and further still more preferably 99% by mass or higher. The heat transfer medium of the present embodiment may contain compounds inert to the compound (A), for example, other compounds functioning as heat transfer media and additives to improve flowability; and although the heat transfer medium contains impurities, it is especially preferable, from the viewpoint of stable temperature control, that the content of the compound (A) is 100% by mass.

The number of carbon atoms of the perfluoroalkenyl group of R^{A} is, from the viewpoint of the flowability as a heat transfer medium, the production easiness and the like, 2 to 4, and preferably 3 or 4. The carbon chain of the perfluoroalkenyl group may be linear or branched, and is preferably linear.

It is preferable, from the viewpoint of the flowability as a heat transfer medium, the production easiness and the like, that either one carbon atom constituting a carbon-carbon double bond is bound to the adjacent oxygen atom.

R^{B} is a fluoroalkyl group having 2 to 4 carbon atoms or a fluoroalkenyl group having 2 to 4 carbon atoms, and is preferably a fluoroalkyl group having 2 to 4 carbon atoms and one hydrogen atom.

In the case where R^{B} is a fluoroalkyl group, the number of carbon atoms thereof is, from the viewpoint of the flowability as a heat transfer medium, the production easiness and the like, 2 to 4, and preferably 3 or 4. The carbon chain of the fluoroalkyl group of R^{B} may be linear or branched, and is preferably linear.

The fluoroalkyl group of R^{B} preferably has one hydrogen atom, and though the hydrogen atom may be bound to any carbon atom, it is preferable that the hydrogen atom is a hydrogen atom (β hydrogen) bound to the second carbon atom from the oxygen atom (etheric oxygen atom) adjacent to R^{B}.

In the case where R^{B} is a fluoroalkenyl group, the number of carbon atoms thereof is, from the viewpoint of the flowability as a heat transfer medium, the production easiness and the like, 2 to 4, and preferably 3 or 4. The carbon chain of the fluoroalkenyl group of R^{B} may be linear or branched, and is preferably linear.

It is preferable, from the viewpoint of the flowability as a heat transfer medium, the production easiness and the like, that either one carbon atom constituting a carbon-carbon double bond in the fluoroalkenyl group of R^{B} is bound to the adjacent oxygen atom.

The fluoroalkenyl group of R^{B} may have a hydrogen atom(s), but is preferably a perfluoroalkenyl group, and preferably the same perfluoroalkenyl group as in R^{A}.

R¹ is an alkylene group having 1 to 4 carbon atoms, preferably 2 to 4, more preferably 2 or 3. The carbon chain thereof may be linear or branched, and is preferably linear.

Specific examples of the compound represented by the formula (1) include 1,2,3,3,3-pentafluoro-1-[2-(1,1,2,3,3,3-hexafluoropropoxy)ethoxy]-1-propene (CF₃CF═CFOCH₂CH₂OCF₂CHFCF₃), 1,2,3,3,3-pentafluoro-1-[3-(1,1,2,3,3,3-hexafluoropropoxy)propoxy]-1-propene (CF₃CF═CFOCH₂CH₂CH₂OCF₂CHFCF₃), and 1,2,3,3,4,4,4-heptafluoro-1-[2-(1,1,2,3,3,4,4,4-octafluorobutoxy)ethoxy]-1-butene (CF₃CF₂CF═CFOCH₂CH₂OCF₂CHFCF₂CF₃). Among these, in consideration of good insulation, the production easiness and the like, preferable is 1,2,3,3,3-pentafluoro-1-[2-(1,1,2,3,3,3-hexafluoropropoxy)ethoxy]-1-propene.

A method for producing the compound (A) is not especially limited, and can be a known method. The compound (A) can be produced by a known synthesis method, for example, a method in which a fluoroolefin and a diol are addition reacted in the presence of an alkali catalyst such as potassium carbonate and thereafter, the resultant is subjected to an elimination reaction of hydrogen fluoride in the presence of a strong base such as an alkali metal alkoxide.

The heat transfer medium of the present embodiment can be used for temperature control of wafers in semiconductor production, cooling and heating of semiconductor devices and electronic parts, cooling of servers, and in heat pumps, heat pipes and thermostatic chambers. The compound (A), since having good insulation, is suitable for cooling or heating of component members in semiconductor production apparatuses, and can meet the severe condition of a high electric field and a high-temperature environment, especially like in a dry etching step using plasma in the semiconductor production.

### EXAMPLES

Hereinafter, the present invention will be described specifically based on Examples, but the present invention is not any more limited to the following Examples.

The identification of reaction products was carried out by using proton nuclear magnetic resonance spectra (1H NMR), fluorine 19 nuclear magnetic resonance spectra (¹⁹F NMR) and gas chromatography mass spectrometry (GC-MS (used column: “DB-1301”, length: 60 m, inner diameter: 250 μm, thickness: 1 μm, manufactured by Agilent Technologies, Inc.).

### [Example 1] Production of Compound 1

In a stainless steel autoclave (internal volume: 2.1 L) equipped with a stirrer, 223 g of potassium carbonate (manufactured by Junsei Chemical Co. Ltd.), 200 g of ethylene glycol (manufactured by Junsei Chemical Co. Ltd.) and 400 g of an anhydrous acetonitrile (manufactured by Fujifilm Wako Pure Chemical Corp.) were charged and held hermetically at 20° C. The content in the autoclave was stirred and 966 g of hexafluoropropene (manufactured by AGC Inc.) in a gas state was added over 6 hours. The resultant was held at 20° C. for 1 hour to be allowed to react, and thereafter, a reaction crude liquid in the autoclave was filtered and recovered.

The recovered reaction crude liquid contained 45% by mass of 1,1,1,2,3,3-hexafluoro-3-[2-(1,1,2,3,3,3-hexafluoropropoxy)ethoxy]propane (HFE (1a)).

Then, In a Hastelloy®-made autoclave (internal volume: 2.1 L), 1,000 g of the reaction crude liquid was charged, and 100 g of an anhydrous hydrogen fluoride (manufactured by AGC Inc.) was added and stirred for 1 hour. An obtained reaction crude liquid was phase separated and washed with water, and dried by Molecular Sieve 3A (manufactured by Fujifilm Wako Pure Chemical Corp.) and thereafter distillation purified to thereby obtain an HFE (1a).

Then, in a glass-made reactor (internal volume: 0.5 L) equipped with a stirrer, 117 g of tert-butyl alcohol (manufactured by Kanto Chemical Co. Inc.) and 75 g of the HFE(1a) were charged and heated at 40° C. under stirring, and 100 g of potassium tert-butoxide (manufactured by Tokyo Chemical Industry Co. Ltd.) was added intermittently over 5 hours. The resultant was held at 40° C. for 2 hours to be allowed to react, and thereafter, a reaction crude liquid was neutralized with hydrochloric acid of 1 mol/L in concentration and washed with water. An organic layer was recovered and distillation purified to thereby obtain 1,2,3,3,3-pentafluoro-1-[2-(1,1,2,3,3,3-hexafluoropropoxy)ethoxy]-1-propene (CF₃CF═CFOCH₂CH₂OCF₂CHFCF₃: Compound 1).

### [Example 2] Production of Compound 2

1,2,3,3,3-pentafluoro-1-[3-(1,1,2,3,3,3-hexafluoropropoxy)propoxy]-1-propene (CF₃CF═CFOCH₂CH₂CH₂OCF₂CHFCF₃): Compound 2) was produced as in Example 1, except for altering ethylene glycol as a synthesis raw material of the HFE(1a) in Example 1 to propylene glycol.

### [Example 3] Production of Compound 3

1,2,3,3,4,4,4-heptafluoro-1-[2-(1,1,2,3,3,4,4,4-octafluorobutoxy)ethoxy]-1-butene (CF₃CF₂CF═CFOCH₂CH₂OCF₂CHFCF₂CF₃: Compound 3) was produced as in Example 1, except for altering hexafluoropropene as a synthesis raw material of the HFE(1a) in Example 1 to octafluorobutene.

### [Evaluation of Physical Properties]

For Compounds 1 to 3, GWP, ODP, the flash point and the dielectric breakdown voltage are shown in Table 1. For comparative reference, the physical properties (catalog values) of Compound 4 are shown as Example 4 together in Table 1, Compound 4 being a commercially available HFE, Novec-7500 (“Novec® 7500”, manufactured by 3M Co.: 3-ethoxy-2-(trifluoromethyl)-1,1,1,2,3,4,4,5,5,6,6,6-dodecafluorohexane (CF₃CF₂CF₂CF(OCH₂CH₃)CF(CF₃)₂).

Then, the GWPs were determined as estimated values as described above. The ODPs were all 0 as described above.

The presence/absence of the flash point was determined by the Tag closed-cup method (JIS K2265-1:2007). No flash point can be decided to be nonflammable.

The dielectric breakdown voltage was measured according to JIS C2101:2100 by a dielectric breakdown tester (“YST-243AT-B100”, manufactured by Yamayo Tester Co., electrode gap: 2.5 mm).

Then, for Compounds 1 to 4, the heat capacity and the heat conductivity were measured to evaluate the heat transfer performance. These results are also shown together in Table 1.

The heat capacity was measured at 25° C. by a differential scanning calorimeter (DSC) (“DSC 8500”, manufactured by PerkinElmer, Inc.).

The heat conductivity was measured at 25° C. with a transient hot-wire device using a platinum wire of 10 μm in wire diameter and 80 mm in length and by such a transient hot-wire method.

TABLE 1

| | | | | | | | Dielectric | Heat | Heat |
|---|---|---|---|---|---|---|---|---|---|
| | | | | | | | Break- | Capacity | Conduc- |
| | | | | | | | down | per Unit | tivity |
| Exam- | | | Classifi- | | | Flash | Voltage | Volume | [mW/ |
| ple | Compound | | cation | GWP | ODP | Point | [kV] | [kJ/(L · K)] | (m · K)] |
| 1 | 1 | CF₃CF═CFOCH₂CH₂OCF₂CHFCF₃ | HFEO | <10*¹ | 0 | absent | 45 | 1.92 | 88.0 |
| 2 | 2 | CF₃CF═CFOCH₂CH₂CH₂OCF₂CHFCF₃ | HFEO | <10*¹ | 0 | absent | 42 | 1.88 | 86.3 |
| 3 | 3 | CF₃CF₂CF═CFOCH₂CH₂OCF₂CHFCF₂CF₃ | HFEO | <10*¹ | 0 | absent | 47 | 1.89 | 84.9 |
| 4 | 4 | Novec-7500 | HFE | 90 | 0 | absent | 35 | 1.82 | 64.7 |
| | | (CF₃CF₂CF₂CF(OCH₂CH₃)CF(CF₃)₂) | | | | | | | |

(remark)
*¹estimated value

As shown in Table 1, the HFEOs of Compounds 1 to 3 had an ODP of 0 and no flash point as in the HFE of Compound 4. Then, the HFEOs had a lower GWP and a higher dielectric breakdown voltage than Compound 4. Hence, it can be decided that Compounds 1 to 3 were low in the environmental load and nonflammable and better in the insulation than Compound 4, which was a conventional heat transfer medium.

Further, the HFEOs of Compounds 1 to 3 had a higher heat capacity and a higher heat conductivity than the HFE of Compound 4, so it can be decided that the HFEOs were heat transfer media excellent in heat transfer performance.

1. A heat transfer medium comprising a compound (A) represented by the following formula (1): 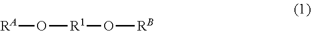
wherein R^{A} is a perfluoroalkenyl group having 2 to 4 carbon atoms; R^{B} is a fluoroalkyl group having 2 to 4 carbon atoms or a fluoroalkenyl group having 2 to 4 carbon atoms; and R¹ is an alkylene group having 1 to 4 carbon atoms.

2. The heat transfer medium according to claim 1, wherein either one carbon atom constituting a carbon-carbon double bond in the perfluoroalkenyl group of R^{A} is bound to the adjacent oxygen atom.

3. The heat transfer medium according to claim 1, wherein either one carbon atom constituting a carbon-carbon double bond in the fluoroalkenyl group of R^{B} is bound to the adjacent oxygen atom.

4. The heat transfer medium according to claim 1, wherein R^{B} is a fluoroalkyl group having 2 to 4 carbon atoms and one hydrogen atom.

5. The heat transfer medium according to claim 1, wherein the compound (A) is 1,2,3,3,3-pentafluoro-1-[2-(1,1,2,3,3,3-hexafluoropropoxy)ethoxy]-1-propene, 1,2,3,3,3-pentafluoro-1-[3-(1,1,2,3,3,3-hexafluoropropoxy)propoxy]-1-propene, or 1,2,3,3,4,4,4-heptafluoro-1-[2-(1,1,2,3,3,4,4,4-octafluorobutoxy)ethoxy]-1-butene.

6. The heat transfer medium according to claim 1, wherein a content of the compound (A) is 50% by mass or higher.

7. The heat transfer medium according to claim 1, wherein the heat transfer medium is used for cooling or heating a component member in a semiconductor production apparatus.
